(19) United States
(12) Patent Application Publication
(10) Pub. No.: US 2006/0073224 A1
(43) Pub. Date: Apr. 6, 2006

(54) Method of preparing Dioscorea tincture and uses thereof

(75) Inventor: Bruce Last, Crestwood, NY (US)

Correspondence Address: NIXON PEABODY LLP - PATENT GROUP, CLINTON SQUARE, P.O. BOX 31051, ROCHESTER, NY, 14603-1051, US

(73) Assignee: Omni Research Technology, Valhalla, NY (US)

(21) Appl. No.: 11/244,942

(22) Filed: Oct. 6, 2005

Related U.S. Application Data
(60) Provisional application No. 60/616,513, filed on Oct. 6, 2004.

(51) Int. Cl. A61K 36/8945

(52) U.S. Cl. 424/773

(57) ABSTRACT

The present invention is directed to method of making a wild yam root tincture from Diascorea sp. and a composition containing this tincture. The composition can be administered to an individual to treat conditions that are treatable with diosgenin.

This application claims the benefit of U.S. Provisional Patent Application Ser. No. 60/616,513, filed Oct. 6, 2004, which is hereby incorporated by reference in its entirety.

### FIELD OF THE INVENTION

The present invention is directed to a method of preparing Dioscorea tincture, compositions containing the same, and uses thereof.

### BACKGROUND OF THE INVENTION

Dioscorea sp. are tropical plants found in many different parts of the world. Although there are well over 150 species on record, there are only a few that demonstrate a high volume of phytohormone, i.e., plant hormone, activity.

The therapeutic effects of Dioscorea sp. date back thousands of years and are connected to numerous cultures. In Chinese medicine it was first mentioned in the Pen Tsao Ching in 26 B.C. The people of the Trobriand Islands near Papua, New Guinea use Dioscorea sp. as a major staple in their diets and exhibit happy, healthy, virile lives unlike those from any other culture. In the USA, Dioscorea sp. has been used since the 1800's in restorative tonics and medicines. Indeed, the phytochemicals known to be present in Dioscorea sp. include beta-carotene, dioscin, dioscorin, diosgenin, niacin, riboflavin, and thiamin. But it was not until the 1940's that U.S. scientists isolated the active components of the Dioscorea sp. root for use in the production of steroidal hormones.

A primary active component of Dioscorea sp. root is the phytohormone diosgenin. The characteristics of the purified crystalline powder of diosgenin include a melting point of around 195° C., a loss of weight upon drying of about 0.5%, and an insolubility in alcohol of about 0.2%.

Diosgenin is a primary material for synthesizing steroid hormone drugs. It is a raw material of 16-dihydropregnenolone acetate. In addition, it can be used in the synthesis of hydrocortisone, prednisone, norethindronum, fluocinolone, dexamethasone, and other steroid hormone drugs.

Compositions comprising solubilized wild yam root extracts, where the compositions are prepared using Dioscorea sp. extracts obtained in powdered form, have been previously shown to have numerous therapeutic effects. Traditionally, manufacturers rely on powdered Dioscorea sp. extract to produce low cost, quickly derived extracts to use in their formulations. To obtain powdered root, wild yam is ground under heated conditions, lyophilized, and stored in drums for unspecified amounts of time until it is needed for processing. Unfortunately, the nature of this extraction process, as well as any subsequent storage of the resulting powdered product, reduces the quality and efficacy of any final compositions containing the extract.

The present invention is directed to overcoming these and other deficiencies in the art.

### SUMMARY OF THE INVENTION

A first aspect of the present invention relates to a method of making a wild yam root tincture. This method includes the steps of: introducing a first batch of wild yam root into a solution comprising water and alcohol, where the first batch of wild yam root is allowed to soak in the solution for about 24 hours or more, removing the first batch of wild yam root from the solution, introducing a second batch of wild yam root into the solution, where the second batch of wild yam root is allowed to soak in the solution for about 24 hours or more, and then removing the second batch of wild yam root from the solution. In this manner, a wild yam root tincture is made.

A second aspect of the present invention relates to a wild yam root tincture produced by the method according to the present invention.

A third aspect of the present invention relates to a composition comprising the tincture according to the second aspect of the invention.

A fourth aspect of the present invention relates to a method of treatment which involves administering to an individual a composition according to the second aspect of the invention, where such administration is effective in treating the subject for a condition selected from the group of, although not limited to, menopause, pre-menstrual syndrome, hormonal imbalance, hot flashes, decreased libido, night sweats, mood swings, bone density deficiency, breast fibroids, eczema, rheumatism and arthritis-like ailments, burns, metabolic regulatory conditions, spasmodic asthma, emotional instability, chronic cough, diarrhea, and diabetes.

The preferred process by which various species of wild yam root, i.e., Dioscorea sp, are extracted yields a high potency diosgenin extract or tincture. The resulting extract is used in the formulation of topically applied emulsions, namely creams and gels. The process of the present invention has been developed to insure that the resulting extract is both fresh and potent.

### BRIEF DESCRIPTION OF THE DRAWINGS

FIG. 1 illustrates the structure of the active ingredient of wild yam root diosgenin.

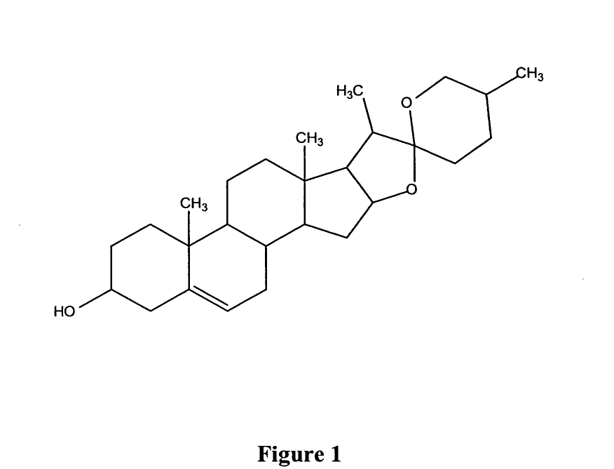

FIG. 2 illustrates schematically one embodiment of the present invention.

### DETAILED DESCRIPTION OF THE INVENTION

The present invention relates to a method of making a wild yam root tincture, use of the tincture in the preparation of topical or oral formulations, and further uses thereof.

One aspect of the present invention relates to a method of making a wild yam root tincture which involves introducing a first batch of wild yam root into a solution comprising water and alcohol, where the first batch of wild yam root is allowed to soak in the solution for about 24 hours or more, removing the first batch of wild yam root from the solution, introducing a second batch of wild yam root into the solution, where the second batch of wild yam root is allowed to soak in the solution for about 24 hours or more, and then removing the second batch of wild yam root from the solution. In this manner, a wild yam root tincture is made.

The method can be performed using fresh whole wild yam root, i.e., Dioscorea sp., preferably Dioscorea villosa, Dioscorea barbasco, Dioscorea bulbifera, or combinations thereof. The first and second batches of wild yam root are preferably freshly chopped prior to introducing them into the solution, where the solution can comprise about 40 to about 80 percent of water or about 50 to about 70 percent of water and about 20 to about 60 percent alcohol or about 30 to about 50 percent alcohol. Preferably, the alcohol is ethanol, although white vinegar can be used as a substitute. The first and second batches of wild yam root can be allowed to soak for up to about 30 days, preferably from about 3 to about 28 days, more preferably from about 7 days or more to about 21 days, at which point (i.e., after the second soak) a wild yam root tincture of the invention is obtained.

Another aspect of the present invention relates to a composition comprising a tincture according to the present invention. The wild yam root tincture is present in an amount effective to achieve a desired remedy. Preferably, the tincture is present in an amount of about 0.05 to about 5.0 percent by weight, or in an amount of about 0.1 to about 2.0 percent by weight.

Tincture obtained from a method of the present invention can be used in a composition that preferably includes one or more carriers. The carrier is preferably any carrier that is suitable for topical application, although other types of carriers for other routes of administration (e.g., oral) are also contemplated.

The carrier can be a water-based fluid carrier that forms an aqueous solution containing the tincture, which may quickly soak into an individual's skin providing for fast transdermal absorption.

The carrier can also be a fluid such as an oil based carrier, a fat based carrier, a fatty alcohol based carrier, or any combination thereof, as described in U.S. Pat. No. 6,573,302 issued to Holt et al., which is hereby incorporated by reference in its entirety.

The carrier can further be an emulsion consisting of an oil phase and a water phase, such as the type described in U.S. Pat. No. 6,730,667 to Deagle, which is hereby incorporated by reference in its entirety. The tincture according to the present invention can be incorporated into the oil phase and water phase and through sheer force a stable emulsion can be obtained. Such an emulsion allows for easy application of the composition to the skin without leaving an oily residue.

According to the present invention, creams and gels can additionally contain various solvents and thickeners, of varying concentrations, depending on the desired consistency and desired properties of the final composition.

Solvents can include, but are not limited to, water, acetic acid, acetone, anisole, 1-butanol, 2-butanol, butyl acetate, tert-butylmethyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, and propyl acetate.

Thickeners and gelling agents can include, but are not limited to, colloidal silicon dioxide, propylene glycol, cetyl alcohol, stearyl alcohol, stearic acid, palmitic acid, glyceryl stearate, triethanolamine, or mixtures thereof The addition of propylene glycol can be used to thicken the gel formulations and to provide a gel that maintains its integrity at body temperature, as described in U.S. Pat. No. 6,365,166 to Beaurline et al., which is hereby incorporated by reference in its entirety.

Compositions can further contain encapsulation agents, colloidal oatmeal, hydrogenated lecithin, dipotassium glycyrrhizinate or a similar encapsulation agent, or even combinations of these agents, as described in U.S. Pat. No. 6,573,302 issued to Holt et al., which is hereby incorporated by reference in its entirety.

Any other known additives can be introduced into creams, gels, or emulsions of the present invention. Exemplary additives include, without limitation, aloe vera gel, zinc compounds, vitamins such as vitamin E, jojoba oil, guar gum, squalane, panthenol, and dimethicone, which can act as moisturizers and skin tissue nutrients.

Administration may be carried out by topical administration to one or more of the buttocks, breasts, underarm, face, neck, and belly and can be self-administered up to several times a day, or as needed by the individual using the composition. The composition can be worked into the skin manually or using a strong massage-type vibrator apparatus.

Administration can also be carried out orally. The composition of the present invention may be orally administered, for example, with an inert diluent, or with an assimilable edible carrier, or it may be enclosed in hard or soft shell capsules. For oral therapeutic administration, the composition may be incorporated with an excipient and used in the form of tablets, capsules, elixirs, suspensions, syrups, and the like. Such compositions and preparations should contain at least 0.01 weight % of active compound. The percentage of the compound in these compositions may, of course, be varied and may conveniently be between about 0.1 to about 10 weight %. The amount of active compound in such therapeutically useful compositions is such that a suitable dosage will be obtained.

When the dosage unit form is a capsule, it may contain, in addition to materials of the above type, a liquid carrier, such as a fatty oil.

A syrup may contain, in addition to active ingredient, sucrose as a sweetening agent, methyl and propylparabens as preservatives, a dye, and flavoring such as cherry or orange flavor.

The present invention also relates to a method of treatment that involves administering to an individual a composition according to the present invention, whereby administering is effective in treating the individual for conditions including, but not limited to, menopause, pre-menstrual syndrome, hormonal imbalance, hot flashes, decreased libido, night sweats, mood swings, bone density deficiency, and breast fibroids. Additional conditions may also be treated according to the methods of the present invention including, but not limited to, eczema, rheumatism and arthritis-like ailments, burns, metabolic regulatory conditions, spasmodic asthma, emotional instability, chronic cough, diarrhea, and diabetes.

In treating one or more of the above conditions, it is contemplated that symptoms of the above-identified conditions can be completely resolved or at least partially resolved (i.e., with continued treatment). For conditions that may moderate over time due to other natural processes or seasonal variation (e.g., burns, diarrhea, eczema, etc.), administration of the composition can be adjusted as needed.

### EXAMPLES

The following examples are provided to illustrate embodiments of the present invention but are by no means intended to limit its scope.

### Example 1

### Preparation of Dioscorea Root Tincture

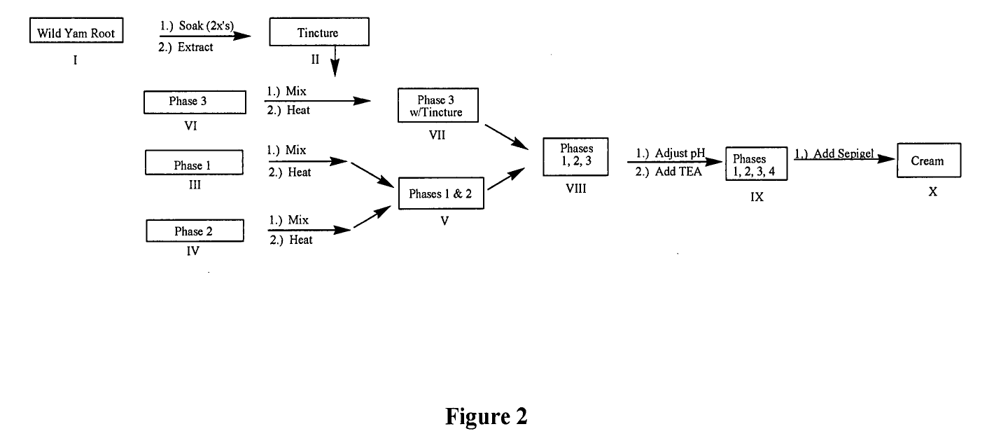

Dioscorea root tincture was prepared using fresh whole wild yam root (I), as illustrated schematically in FIG. 2. Within about 30 seconds of freshly chopping the roots, the roots were immediately added to a water/alcohol menstrum containing 60 percent water and 40 percent ethanol. The roots were covered with the menstrum and left to soak for 21 days. After soaking, the roots were removed and discarded, and new freshly chopped roots were added to the menstrum. This second extraction was soaked for an additional 21 days. Upon subsequent removal of the roots, the tincture (II) was obtained.

### Example 2

### Preparation of Topical Cream Containing Tincture

A topical cream containing the tincture was prepared by preparing and then blending five distinct phases, as is illustrated schematically in FIG. 2.

Phase 1: Phase 1 materials (III) were weighed and added to a suitable vessel. Phase 1 materials included: 65.4260 weight % of deoionized water, 0.1860 weight % of Aloe Vera Gel/Activgel in a 1:1 ratio, 0.2000 weight % of Methyl Paraben N.F., 1.2000 weight % of 99.7% Glycerine USP, 0.280 weight % of Grape Seed Powder (Ext.), 2.5000 weight % of 99% N5 Triethanolamine Liquid, and 0.1000 weight % of Trisodium EDTA. The materials were heated to 75° C. and mixed. Maintaining the mixture at 75° C., it was then mixed until uniform conditions were achieved.

Phase 2: In a separate vessel, phase 2 materials (IV) were carefully heated to 75° C., making sure that the mixture was not overheated. Phase 2 materials included: 5.6300 weight % of Cetyl Alcohol NF, 10.0500 weight % of Soybean Oil U.S.P., 6.0000 weight % of Stearic Acid NF EXFLAKE, 0.1000 weight % of Propyl Paraben N.F., and 1.1000 weight % of Vitamin E Acetate USP DL-Alpha. The materials were mixed until they were uniform, added to the main vessel containing the phase 1 materials, then homomixed for 10 minutes at 75° C. Homomixing was stopped and the mix sweeped while cooled to 40° C. to produce an intermediate composition (V).

Phase 3: Phase 3 materials (VI) were mixed thoroughly and kept away from extreme heat, sparks, and open flames. Phase 3 materials included: 4.1900 weight % of Deionized water, 0.1000 weight % of A35 Wild Yam Tincture (II) prepared according to Example 1, 0.0500 weight % of fine granula Ascorbic Acid, 0.3000 weight % of Germall 115, and 1.3400 weight % of 190 Proof SDA 40B Alcohol. Phase 3 materials with tincture (VII) were then added to the vessel containing the phase 1 and phase 2 materials (V). The resulting mixture (VIII) was mixed until uniform, and cooled to 25° C.

Phase 4: The mixture in the vessel containing phases 1-3 material (VIII) was adjusted to a pH of between about 7.5-8.5, and then 0.5000 weight % of 99% N5 Triethanolamine (“TEA”) was added in increments. The materials in the vessel were then mixed until uniform (IX).

Phase 5: 0.7480 weight % of Sepigel 305™ was added to the mixture and mixed by a recirculation method at 25° C. until uniform. The final product (X) was milled through a colloidal mill at 25-30° C. and a cream according to the present invention was obtained.

The finished product sample was then submitted to Quality Control (“QC”) for testing. The resulting cream had a faint characteristic odor, a smooth uniform opaque cream appearance, a pH at 25° C. of approximately 7.5-8.5, a specific gravity at 25° C. of 0.94±0.02, and a viscosity of 55-65 centipoise at 25° C.

Using the extraction process of the present invention, creams with high levels of the steroidal saponin diosgenin were produced averaging >0.02 wt % of diosgenin. In contrast, creams prepared from powdered root extract (as made by Natural EFX of Dallas, Tex.) yielded levels of diosgenin averaging <0.01 wt %. Diosgenin is a known hormone balancer and/or regulator, thus the higher diosgenin levels present in creams of the present invention are believed to have the ability to more efficiently treat conditions known to respond to diosgenin therapy.

### Example 3

### Treatment of Hormonal-Associated Symptoms

Patients suffering from symptoms associated with their hormone cycles, including but not limited to hot flashes, will typically massage ¼ to ½ teaspoon of the cream described in Example 2 onto clean skin, particularly the skin of the inner arms, upper chest and abdomen, thighs, and buttocks. The cream should be applied twice daily for 21 days. Use may be discontinued for 7 days, then the cycle of administration repeated as needed. In addition, the site of application can be rotated to ensure maximal absorption of the diosgenin. To further facilitate product absorption and to protect against dehydration, fluid intake should be increased when using the cream.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of making a Wild Yam root tincture, said method comprising:
introducing a first batch of Wild Yam root into a solution comprising water and alcohol, wherein the first batch of Wild Yam root is allowed to soak in the solution for about 24 hours or more;
removing the first batch of Wild Yam root from the solution; and
introducing a second batch of Wild Yam root into the solution, wherein the second batch of Wild Yam root is allowed to soak in the solution for about 24 hours or more; and
removing the second batch of Wild Yam root from the solution, whereby a Wild Yam root tincture is made.

2. The method according to claim 1, wherein the Wild Yam root is Dioscorea villosa, Dioscorea barbasco, Dioscorea bulbifera, or a combination thereof.

3. The method according to claim 1, wherein the first and second batches of Wild Yam root are freshly chopped prior to said introducing.

4. The method according to claim 1, wherein the solution comprises about 40 to about 80 percent of water.

5. The method according to claim 1, wherein the solution comprises about 50 to about 70 percent of water.

6. The method according to claim 1, wherein the alcohol is ethanol.

7. The method according to claim 1, wherein the solution comprises about 20 to about 60 percent alcohol.

8. The method according to claim 1, wherein the solution comprises about 30 to about 50 percent alcohol.

9. The method according to claim 1, wherein the first and second batches of Wild Yam root are allowed to soak for about 7 days or more.

10. The method according to claim 1, wherein the first and second batches of Wild Yam root are allowed to soak for about 21 days.

11. A Wild Yam root tincture produced by the method according to claim 1.

12. The Wild Yam root tincture according to claim 1 1, wherein the Wild Yam root is Dioscorea villosa, Dioscorea barbasco, Dioscorea bulbifera, or a combination thereof.

13. A composition comprising the Wild Yam root tincture according to claim 11.

14. The composition according to claim 13, wherein the composition is a cream or gel.

15. The composition according to claim 13 further comprising a carrier.

16. The composition according to claim 11, wherein the Wild Yam room tincture is present in an amount of about 0.05 to about 5 percent by weight.

17. The composition according to claim 11, wherein the Wild Yam room tincture is present in an amount of about 0.1 to about 2 percent by weight.

18. A method of treatment comprising:
administering to an individual a composition according to claim 11, whereby said administering is effective to treat the individual for a condition selected from the group of menopause, pre-menstrual syndrome, hormonal imbalance, hot flashes, decreased libido, night sweats, mood swings, bone density deficiency, breast fibroids, eczema, rheumatism and arthritis-like ailments, burns, metabolic regulatory conditions, spasmodic asthma, emotional instability, chronic cough, diarrhea, and diabetes.

19. The method according to claim 18, wherein said administering is carried out by topical administration or oral administration.

20. The method according to claim 19, wherein said topical administration comprises applying the composition to one or more of the buttocks, breasts, underarm, face, neck, and belly.
